(19) United States
(12) Patent Application Publication
(10) Pub. No.: US 2024/0271079 A1
(43) Pub. Date: Aug. 15, 2024

(54) CULTURED BUFFALO MILK PRODUCTION METHODS, SYSTEMS, COMPOSITIONS AND USES THEREOF

(71) Applicants: Bee-io Honey Technologies LTD., Rehovot (IL); THE IP LAW FIRM OF GUY LEVI, LLC, Wyckoff, NJ (US)

(72) Inventors: Efrat Dvash Riesenfeld, Hatzav (IL); Ofir Dvash, Givatayim (IL)

(21) Appl. No.: 18/285,703

(22) PCT Filed: Jun. 2, 2023
(86) PCT No.: PCT/US23/24277

§ 371 Date: Oct. 5, 2023

Related U.S. Application Data
(60) Provisional application No. 63/348,386, filed on Jun. 2, 2022.

(51) Int. Cl. C12M 1/00; A23C 23/00; A23J 1/00; C12M 1/02; C12M 3/00

(52) U.S. Cl. CPC C12M 47/10; A23C 23/00; A23J 1/008; C12M 23/44; C12M 27/00; C12M 47/12

(57) ABSTRACT

The disclosure relates to methods, systems and compositions for producing Buffalo milk, milk proteins and milk products. More specifically, the disclosure relates to methods systems, and compositions for continuously, batch-wise and semi-continuously produce Buffalo milk, milk proteins and milk products using transformed/transfected yeast and/or fungi and/or bacteria and/or algea cultured in bioreactors and/or fermenters, to express and/or secrete Bubalus bubalis Casein, Whey protein and additional proteins, collecting the expressed product and using it for producing various products.

### BACKGROUND

The disclosure is directed to methods, systems and compositions for producing Buffalo milk, milk proteins and milk products. More specifically, the disclosure is directed to methods systems, and compositions for continuously, batch-wise and semi-continuously produce Buffalo milk, milk proteins and milk products using transformed/transfected yeast and/or fungi and/or bacteria and/or algea, and/or mammary epithelial cells cultured in bioreactors and/or fermenters, to express and/or secrete Bubalus bubalis Casein, Whey protein and additional proteins, collecting the expressed product and using it for producing various products.

Existing milk alternatives, such as soy, and oat milk fall short both in flavor and in functionality; moreover, a large part of the industrial and cultural significance of dairy milk stems from its usefulness in derivative products, such as cheese, yogurt, cream, or butter. Non-dairy plant-based milks, while addressing environmental and health concerns, almost universally fail to form such derivative products when subjected to the same processes used for dairy milk.

Moreover, recent report from IATP noted, that as of 2017, the 13 top dairy companies' emissions grew 11% compared with 2015, corresponding to a 32.3 million metric ton increase in greenhouse gases equivalent to the emissions that would be released by adding an extra 6.9 million cars to the road for a year.

Buffalo milk is the second most consumed milk worldwide, representing 15% of the world milk production (˜910 million tons) with an annual growth rate of ˜2.5%. (Vargas-Ramella et al., 2021). Buffalo milk is characterized by a rich composition with high content of fatty acids and proteins. Moreover, it is a good source of vitamins A, D, C and B6, minerals such as calcium and phosphorus, and conjugated linoleic acid (Pasquini et al., 2018). A recent study, highlighted that buffalo milk proteins are less allergenic than cow milk proteins (Ahmed., 2013; Kapila et al., 2013)

Buffalo milk dairy products are known for their high-quality. Among these are pasteurized or concentrated milk, butter, heat-desiccated dairy products, heat acid coagulated dairy products, yogurt, ice-cream, dehydrated milk products and cheeses. The Mozzarella cheese, made from the milk of Italian Mediterranean buffalo, with protected designation of origin mark is the most famous in the world. Due to the high nutritional value of buffalo milk, the demand for such products is increasing (Cazacu et al., 2014; D'Ambrosio et al., 2008; Pasquini et al., 2018).

The following addresses and combines the shortcomings of the current milk alternatives, the demand and the environmental concerns, with the benefits of Buffalo milk products.

### SUMMARY

Disclosed, in various implementations, are methods, systems and compositions for producing cultured Buffalo milk, milk proteins, and milk products. More specifically, provided herein are exemplary implementations of methods, systems, compositions for continuously, batch-wise and semi-continuously producing cultured buffalo milk, milk proteins and milk products using transformed/transfected yeast and/or bacteria and/or fungi and/or algea and/or mammry epithelial cells (MEC), cultured in fermenters/bioreactors to express Buffalo milk, milk proteins, and milk products, collecting the expressed products and their use in edible and other products.

In an exemplary implementation, provided herein is a system for producing Bubalus bubalis cultured milk, milk proteins and milk products, the system comprising: a plurality of bioreactors, each having a proximal end and a distal end, each bioreactor further containing the at least one of recombinant: a yeast, a bacterium, a fungus, and an algae comprising heterologous polynucleotides encoding a Bubalus bubalis polypeptide, wherein each bioreactor is further being in liquid communication with a mixing tank; and a plurality of collection receptacle, each collection receptacle associated with a bioreactor. In certain implementation, each protein in the plurality of vessels, is expressed and isolated individually, then collected for further use. The proteins sought to be isolated, can be purified using a purification module comprising for example at least one of: ÄKTA™ automated system (with or without an autosampler), an HPLC with affinity column, and a sup concentrator.

In another exemplary implementation, the systems disclosed further comprise a bioreactor comprising a carrier having thereon Bubalus bubalis mammary epithelial cells (MECs), adapted to secrete and accumulate a fatty acid, or a carrier having thereon a recombinant yeast adapted to overproduce extra-cellular free fatty acids (FFAs), or an oleaginous microorganism such as, fungi, yeasts or algae that accumulate high levels of lipids and free fatty acids, wherein the bioreactor comprising the Bubalus bubalis MECs, or the recombinant yeast, or the oleaginous microorganism is in liquid communication with the mixing tank, and a FFA separator, the FFA separator being in further in liquid communication with the mixing tank.

In yet another exemplary implementation, the system further comprises a bioreactor comprising a carrier having thereon a recombinant yeast comprising heterologous polynucleotides encoding proteins required for human milk oligosaccharide (HMO) biosynthesis, wherein the bioreactor comprising the recombinant yeast is in liquid communication with the mixing tank, and a HMO separator, the HMO separator being in further liquid communication with the mixing tank.

### BRIEF DESCRIPTION OF THE FIGURES

For a better understanding of the methods, systems, compositions for continuously, batch-wise and semi-continuously using transformed/transfected yeast and/or fungi and/or bacteria and/or algea to express and/or secrete Bubalus bubalis Casein, Whey protein and additional proteins, collecting the expressed product and using it for producing various products, reference is made to the following drawing(s), in which:

FIG. 1, is a schematic illustrating an exemplary implementation of the bioreactors configuration for production of cultured buffalo milk, milk proteins and milk products.

### DETAILED DESCRIPTION

Provided herein are exemplary implementations of methods, systems and compositions for producing cultured Buffalo milk, milk proteins, and milk products. More specifically, provided herein are exemplary implementations of methods, systems, compositions for continuously, batch-wise and semi-continuously producing cultured buffalo milk using transformed/transfected yeast and/or bacteria and/or fungi and/or algea and/or mammry epithelial cells (MEC) cultured in fermenters/bioreactors to express Buffalo milk, milk proteins, and milk products, collecting the expressed products and their use in edible and other products. The process is sustainable, cost effective with high production capacity and without harming any animals. The cultured buffalo milk and products produced will be free of antibiotics, growth hormones, or pesticides common in milk nowdays, and as such will answer a growing costumer demand for clean products.

Buffalo milk is characterized by a rich composition with high content of fatty acids and proteins. For example, it was found that the Mean energy (107 kcal/100 g), protein (4.5 g/100 g), and fat (7.6 g/100 g) concentrations in water buffalo milk were 51%, 29%, and 85% higher than reported in Egyptian dairy cow's milk, respectively (Linfesty L., 2020). Moreover, Buffalo milk is a good source of vitamins A, D, C and B6, and minerals such as calcium and phosphorus, and conjugated linoleic acid. The presence of trace elements such as boron, cobalt, copper, iron, manganese, sulfur, and zinc has also been ascertained (Pasquini et al., 2018). Buffalo milk and milk components can provide in certain cases health benefits to subjects in need thereof, suffering from hypertension, dental decay, dehydration, respiratory problems, obesity, osteoporosis and some forms of cancer. Furthermore, it was found that buffalo milk proteins are less allergenic than cow milk proteins (Ahmed., 2013; Kapila et al., 2013).

In ruminant's milk, αS1-casein(αS1-CN), αS2-casein (αS2-CN), β-casein (β-CN), κ-casein (κ-CN), β-lactoglobulin (β-LGB) and α-lactalbumin B (α-LAB) are the major milk proteins. Therefore, the first step in producing cultured buffalo milk is to obtain buffalo milk proteins via microbial and/or the use of buffalo mammary epithelial cells expression systems.

Moreover, the percentage of casein in milk as a raw material, determines cheese yield. Therefore, changing milk composition for higher casein percentage has been in great demand by the dairy industry. An increase of 20% in the content of αS1-casein of milk could result in an increase of $200 million per year. Milk proteins could also be exploited as in the manufacturing of milk protein concentrates (MPC). Typically, edible casein is being used in vitamin tablets, instant drinks and infant formulae. Technical acid caseins are used for paper coatings, cosmetics, button making, paints and textile fabrics (Sukla et al., 2007).

Moreover, β-casein has two variants: A1 and A2. Some people have difficulties digesting β-casein A1, which can cause gastrointestinal disorders (and discomfort). A2 milk, which contains only β-casein A2, is considered a viable alternative. A study aimed to evaluate the alleles of β-casein in Buffaloes had shown that A1 does not exist in Buffalo species. Thus, all milk products of Buffaloes are naturally A2, adding value to products derived from Buffalo milk (de Oliveira et al., 2021).

Whey proteins (β-lactoglobulin, and α-lactalbumin B), which represent 20% of the total protein contained in milk are used as an active food ingredient in the production of functional foods and in broad applications, for example as an encapsulating agent or carrier materials to entrap bioactive compounds, for emulsification, and in edible and active packaging. Moreover, biological activities of both the intact proteins, and peptides derived from these proteins (in other words; β-lactoglobulin, and α-lactalbumin B), include inhibition of angiotensin-converting enzyme (non-selective ACE inhibitors), anti-microbial activity, anti-carcinogenic activity, anti-hypocholesterolemic effect, as well as having beneficial metabolic and physiological effects (Chatterton et al., 2006). Bassan et al., 2015 had shown that the amount of released amino acids from Buffalo whey is much higher than the amount released from bovine whey making it a better source of bioavailable amino acids.

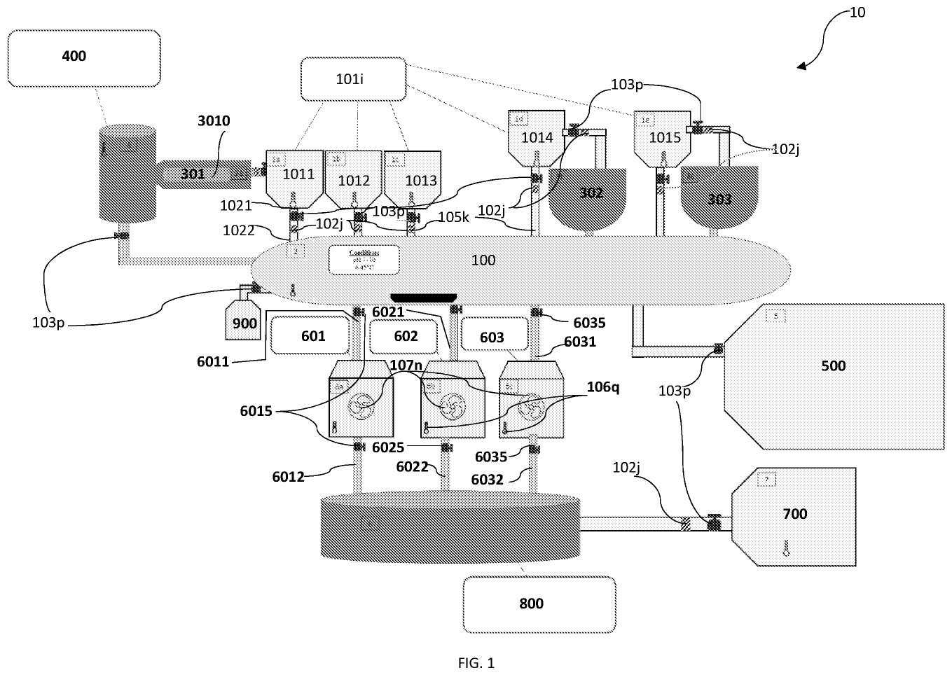

Accordingly and in an exemplary implementation illustrated schematically in FIG. 1, provided herein is system 10 for producing Bubalus bubalis milk product, the system comprising: plurality of bioreactors 101i, e.g., 1011, 1012, 1013, each i^{th} bioreactor having proximal end (e.g., 1021) and distal end (e.g., 1022), each i^{th} bioreactor further containing at least one of a recombinant: a yeast, a bacterium, a fungus, and an algae, comprising heterologous polynucleotides encoding a Bubalus bubalis polypeptide, wherein each i^{th} bioreactor is further being in liquid communication with mixing tank 100; and plurality of collection receptacles 601, 602, 603, each collection receptacle 601, 602, 603, associated with a corresponding combination of bioreactors 101i, depending on the product sought to be produced. For example, Standardized milk will contain proteins from all three bioreactors 1011, 1012, 1013. Crème will be collected from bioreactors 1011, 1012, 1013 with higher fat percentage from separator 302, while whey will contain only whey proteins collected in an exemplary implementation from bioreactor 1012 only. Accordingly, in an exemplary implementation, bioreactor 1011, is operable to express milk proteins, and bioreactor 1013, is operable to express mature milk antimicrobial proteins as described herein

In the context of the disclosure, the term “recombinant” refers to a non-naturally occurring DNA, protein, cell, seed, or organism that is the result of genetic engineering and as such would not normally be found in nature. A “recombinant DNA molecule” is a DNA molecule comprising a DNA sequence that does not naturally occur in nature and as such is the result of human intervention, such as a DNA molecule comprised of at least two DNA molecules heterologous to each other. An example of a recombinant DNA molecule is a DNA molecule provided herein encoding a Bubalus bubalis protein component operably linked to a heterologous regulatory or other element, such as a heterologous promoter for expression in at least one of: a yeast, a bacterium, a fungus, and an algae. A “recombinant protein” is a protein comprising an amino acid sequence that does not naturally occur and as such is the result of human intervention, such as an engineered protein or a chimeric protein. A recombinant cell, or organism is a cell, or organism comprising transgenic DNA, for example a transgenic cell, or organism comprising a recombinant DNA molecule and therefore produced as a result of transformation.

Likewise, and in the context of the disclosure, the term “transformed”, “transform” or “transformation” refers to transient, stable or permanent changes in the characteristics (expressed phenotype) of a cell by the mechanism of gene transfer. Genetic material is introduced into a cell in a form where it expresses a specific gene product or alters the expression or effect of endogenous gene products. Transformation can occur via various mechanisms such as transfection, electroporation or particle bombardment. As used herein the term “transfected” or “transfection” refers to the incorporation of foreign DNA into cultured cells by exposing them to such DNA. This would include the introduction of DNA by various delivery methods, e.g., via vectors or plasmids Following entry into the cell, the transfected DNA may: (1) recombine with that of the host; (2) replicate independently as a plasmid or temperate phage; or (3) be maintained as an episome without replication prior to elimination.

The terms “polypeptide” and “protein” are used interchangeably herein to refer to a polymer of consecutive amino acid residues. Moreover, in the context of the disclosure, the term “nucleic acid”, “nucleotide”, and “polynucleotide” are used interchangeably and refer to RNA, DNA, cDNA, or cRNA and derivatives thereof, such as those containing modified backbones. It should be appreciated that the polynucleotides comprising sequences complementary to those described herein are also contemplated. The “polynucleotide” contemplated herein includes both the forward strand (5′ to 3′) and reverse complementary strand (3′ to 5′). Polynucleotides disclosed can be prepared in different ways (e.g. by chemical synthesis, by gene cloning etc.) and can take various forms (e.g. linear or branched, single or double stranded, or a hybrid thereof, primers, probes etc.). The term “heterologous DNA”, or “heterologous polynucleotide” as used herein refers to the DNA derived from a different organism, such as a different cell type or a different species from the recipient. The term also refers a DNA or fragment thereof on the same genome of the host DNA wherein the heterologous DNA is inserted into a region of the genome which is different from its original location.

The Bubalus bubalis polypeptide, expressed, isolated and collected using the systems disclosed can be at least one of: a milk protein, a whey protein, and an antimicrobial protein. For example, the milk protein is at least one of: αS1-casein (αS1-CN), αS2-casein (αS2-CN), β-casein (β-CN), and κ-casein (κ-CN) encoded by the following sequences:

| SEQ ID | | | |
|---|---|---|---|
| No. | Sequence | Name | Encodes |
| 1 | ATGAAACTTCTCATCCTTACCTGTCTTGTGGCTGTTGCTCTTGCCAGGCCT | α-S1 casein | DQ111783.1 |
| | AAACAGCCTATCAAGCACCAAGGACTCCCTCAAGGAGTCCTCAATGAAA | | |
| | ATTTACTCAGGTTTTTTGTGGCGCCTTTTCCAGAAGTGTTTGGAAAGGAGA | | |
| | AGGTCAATGAACTGAGCACGGATATTGGGAGTGAATCAACTGAGGATCA | | |
| | AGCCATGGAAGATATTAAGCAAATGGAAGCTGAAAGCATTTCGTCAAGT | | |
| | GAGGAAATTGTTCCCATTAGTGTTGAGCAGAAGCACATTCAAAAGGAAG | | |
| | ATGTGCCCTCTGAGCGTTACCTGGGTTATCTGGAACAGCTTCTTAGACTG | | |
| | AAAAAATACAACGTACCCCAGCTGGAAATTGTTCCCAATTTGGCTGAGGA | | |
| | ACAACTTCACAGTATGAAAGAGGGAATCCATGCCCAACAGAAAGAACCT | | |
| | ATGATAGGAGTGAATCAGGAACTGGCCTACTTCTACCCTCAGCTTTTCAG | | |
| | ACAATTCTACCAGCTGGACGCCTATCCATCTGGTGCCTGGTATTACGTTCC | | |
| | ACTAGGCACGCAATACCCTGATGCCCCATCATTCTCTGACATCCCTAATC | | |
| | CCATCGGCTCTGAGAACAGTGAAAAGACTACTATGCCACTGTGGTGA | | |
| 2 | MKLLILTCLVAVALARPKQPIKHQGLPQGVLNENLLRFFVAPFPEMFGKEKV | | QPO15022.1 |
| | NELSTDVGSESTEDQAMEDIKQMEAESISSSEEIVPISVEQKHIQKEDVPSERY | | |
| | LGYLEQLLRLKKYNVPQLEIVPNLAEEQLHSMKEGIHAQQKEPMIGVNQELA | | |
| | YFYPQLFRQFYQLDAYPSGAWYYVPLGTQYPDAPLESDIPNPIGSENSGKTT | | |
| | MPLW | | |
| 3 | ATGAAGTTCTTCATCTTTACCTGCCTTTTGGCTGTTGCCCTTGCAAAGCAT | α-s2 casein | DQ173244.1 |
| | ACGATGGAACATGTCTCCTCCAGTGAGGAATCTATCATCTCCCAGGAAAC | | |
| | ATATAAGCAGGAAAAGAATATGGCCATTCATCCCAGCAAGGAGAACCTT | | |
| | TGCTCCACATTCTGCAAGGAAGTTATAAGGAATGCAAATGAAGAGGAAT | | |
| | ATTCTATCGGCTCATCTAGTGAGGAATCTGCTGAAGTTGCCACAGAGGAA | | |
| | GTTAAGATTACTGTGGACGATAAGCACTACCAGAAAGCACTGAATGAAA | | |
| | TCAATCAGTTTTATCAGAAGTTCCCCCAGTATCTCCAGTATCTGTATCAAG | | |
| | GTCCAATTGTTTTGAACCCATGGGATCAGGTTAAGAGAAATGCTGTTCCC | | |
| | ATTACTCCCACTCTGAACAGAGAACAGCTCTCCACCAGTGAGGAAAATTC | | |
| | AAAGAAGACCGTTGACATGGAATCAACAGAAGTAATCACTAAGAAAACT | | |
| | AAACTGACTGAAGAAGATAAGAATCGCCTAAATTTTCTGAAAAAAATCA | | |
| | GCCAGCATTACCAGAAATTCACCTGGCCCCAGTATCTCAAGACTGTTTAT | | |
| | CAGTATCAGAAAGCTATGAAGCCATGGACTCAACCTAAGACAAAGGTTA | | |
| | TTCCCTATGTGAGGTACCTTTAA | | |
| 4 | MKFFIFTCLLAVALAKHTMEHVSSSEESIISQETYKQEKNMAIHPSKENLCSTF | | AAZ80050.1 |
| | CKEVIRNANEEEYSIGSSSEESAEVATEEVKITVDDKHYQKALNEINQFYQKF | | |
| | PQYLQYLYQGPIVLNPWDQVKRNAVPITPTLNREQLSTSEENSKKTVDMEST | | |
| | EVITKKTKLTEEDKNRLNFLKKISQHYQKFTWPQYLKTVYQYQKAMKPWTQ | | |
| | PKTKVIPYVRYL | | |
| 5 | ATGAAGGTCCTCATCCTTGCCTGCCTGGTGGCTCTGGCCCTTGCAAGAGA | 2 casein | MN560175.1 |
| | GCTGGAAGAACTCAATGTACCCGGTGAGATTGTGGAAAGCCTTTCAAGCA | beta (CSN2) | |
| | GTGAGGAATCTATTACACACATCAATAAGAAAATTGAGAAGTTTCAGAGT | | |
| | GAGGAACAGCAGCAAATGGAGGATGAACTCCAGGATAAAATCCACCCCT | | |
| | TTGCCCAGACACAGTCTCTAGTCTATCCCTTCCCTGGGCCCATCCCTAAGA | | |
| | GCCTCCCACAAAACATCCCGCCTCTTACTCAAACCCCTGTGGTGGTGCCG | | |
| | CCTTTCCTTCAGCCTGAAATAATGGGAGTCTCCAAAGTGAAGGAGGCTAT | | |
| | GGCTCCTAAGCACAAAGAAATGCCCTTCCCTAAATATCCAGTTGAGCCCT | | |
| | TTACTGAAAGCCAGAGCCTGACTCTCACTGATGTTGAAAATCTGCACCTT | | |
| | CCTCTGCCTCTGCTCCAGTCTTGGATGCACCAGCCTCCCCAGCCTCTGCCT | | |
| | CCAACTGTCATGTTTCCCCCTCAGTCCGTGCTGTCCCTTTCTCAGTCCAAA | | |
| | GTTCTGCCTGTTCCCCAGAAAGCAGTGCCCTATCCCCAGAGAGATATGCC | | |
| | CATTCAGGCCTTTCTGCTGTACCAGGAGCCTGTACTTGGTCCTGTCCGGGG | | |
| | ACCCTTCCCTATTATTGTCTAA | | |
| 6 | MKVLILACLVALALARELEELNVPGEIVESLSSSEESITHINKKIEKFQSEEQQ | | CAZ66675.1 |
| | QMEDELQDKIHPFAQTQSLVYPFPGPIPKSLPQNIPPLTQTPVVVPPFLQPEIM | | |
| | GVSKVKEAMAPKHKEMPFPKYPVEPFTESQSLTLTDVENLHLPLPLLQSWM | | |
| | HQPPQPLPPTVMFPPQSVLSLSQSKVLPVPQKAVPYPQRDMPIQAFLLYQEPV | | |
| | LGPVRGPFPIIV | | |
| 7 | ATGATGAAGAGTTTTTTCCTAGTTGTGACTATCCTGGCATTAACCCTGCCA | k-casein | AY750857.1 |
| | TTTTTGGGTGCCCAGGAGCAAAACCAAGAACAACCAATACGCTGTGAGA | | |
| | AAGAGGAAAGATTCTTCAATGACAAAATAGCCAAATATATCCCAATTCA | | |
| | GTATGTGCTGAGTAGGTATCCTAGTTATGGACTCAATTACTACCAACAGA | | |
| | AACCAGTTGCACTAATTAATAATCAATTTCTGCCATACCCATATTATGCA | | |
| | AAGCCAGCTGCAGTTAGGTCACCTGCCCAAATTCTTCAATGGCAAGTTTT | | |
| | GCCAAATACTGTGCCTGCCAAGTCCTGCCAAGCCCAGCCAACTACCATGA | | |
| | CACGTCACCCACACCCACATTTATCATTTATGGCCATTCCACCAAAGAAA | | |
| | AATCAGGATAAAACAGAAATCCCTACCATCAATACCATTGTTAGTGTTGA | | |
| | GCCTACAAGTACACCTACCACCGAAGCAATAGAGAACACTGTAGCTACTC | | |
| | TAGAAGCTTCCTCAGAAGTTATTGAGAGTGTACCTGAGACCAACACAGCC | | |
| | CAAGTTACTTCAACCGTCGTCTAA | | |
| 8 | MMKSFFLVVTILALTLPFLGAQEQNQEQPIRCEKEERFFNDKIAKYIPIQYVLS | | AAU95771.1 |
| | RYPSYGLNYYQQKPVALINNQFLPYPYYAKPAAVRSPAQILQWQVLPNTVP | | |
| | AKSCQAQPTTMTRHPHPHLSFMAIPPKKNQDKTEIPTINTIVSVEPTSTPTTEAI | | |
| | ENTVATLEASSEVIESVPETNTAQVTSTVV | | |

Or a sequence having between 80% and 99% homology, its mRNA and cDNA or showing the activity of the encoded proteins. Moreover, the phrase “nucleic acid sequence” as used herein refers to a consecutive list of abbreviations, letters, characters or words, which represent nucleotides. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”.

Likewise, Bubalus bubalis polypeptide, expressed, isolated and collected using the systems disclosed can be whey protein, that is at least one of: β-lactoglobulin (β-LGB) and α-lactalbumin B (α-LAB), encoded by the following sequences:

| SEQ ID | | | |
|---|---|---|---|
| No. | Sequence | Name | Encodes |
| 9 | ATGATGTCCTTTGTCTCTCTGCTCCTGGTAGGCAG | α-lactalbumin | DQ785796.1 |
| | CCTATTCCATGCCACCCAGGCAGAACAATTAACA | | |
| | AAATGTGAGGTGTTCCGGGAGCTGAGAGACTTG | | |
| | AAGGACTACGGAGGTGTCAGTTTGCCTGAATGGG | | |
| | TCTGTACCGCGTTTCATACCAGTGGTTATGACAC | | |
| | ACAAGCCATAGTACAAAACAATGACAGCACAGA | | |
| | ATATGGACTCTTCCAGATAAATAATAAAATTTGG | | |
| | TGCAAAGACGACCAGAACCCTCACTCAAGCAAC | | |
| | ATCTGTAACATCTCCTGTGACAAGTTCCTGGATG | | |
| | ATGATCTTACTGATGACATTATGTGTGTCAAGAA | | |
| | GATTCTGGATAAAGTAGGAATTAACTACTGGTTG | | |
| | GCCCATAAAGCACTCTGTTCTGAGAAGCTGGATC | | |
| | AGTGGCTCTGTGAGAAGTTGTGA | | |
| 10 | MMSFVSLLLVGSLFHATQAEQLTKCEVFRELRDLK | | ABG78269.1 |
| | DYGGVSLPEWVCTAFHTSGYDTQAIVQNNDSTEY | | |
| | GLFQINNKIWCKDDQNPHSSNICNISCDKFLDDDLT | | |
| | DDIMCVKKILDKVGINYWLAHKALCSEKLDQWLC | | |
| | EKL | | |
| 11 | ATGAAGTGCCTCTTGCTTGCCCTGGGCCTGGCCC | β-lactoglobulin | AJ005429.1 |
| | TTGCCTGTGCGGCCCAGGCCATCATCGTCACCCA | | |
| | GACCATGAAGGGCCTGGACATCCAGAAGGTGGC | | |
| | GGGGACTTGGTACTCCTTGGCCATGGCGGCCAGC | | |
| | GACATCTCCCTGCTGGACGCCCAGAGTGCCCCCC | | |
| | TGAGAGTGTATGTGGAGGAGCTGAAGCCCACCC | | |
| | CTGAGGGCGACCTGGAGATCCTGCTGCAGAAAT | | |
| | GGGAGAATGGTGAGTGTGCTCAGAAGAAGATCA | | |
| | TTGCAGAAAAAACCAAGATCCCTGCCGTGTTCAA | | |
| | GATCGACGCCTTGAACGAGAACAAAGTCCTTGTG | | |
| | CTGGACACCGACTACAAAAAGTACCTGCTCTTCT | | |
| | GCATGGAGAACAGTGCTGAGCCCGAGCAAAGCC | | |
| | TGGCCTGCCAGTGCCTGGTCAGGACCCCGGAGGT | | |
| | GGACGACGAGGCCCTGGAGAAATTTGACAAAGC | | |
| | CCTCAAGGCCCTGCCTATGCACATCCGGCTCTCC | | |
| | TTCAACCCGACCCAGCTGGAGGAGCAGTGCCAC | | |
| | GTCTAG | | |
| 12 | MKCLLLALGLALACGAQAIIVTQTMKGLDIQKVAG | | CAY39357.1 |
| | TWYSLAMAASDISLLDAQSAPLRVYVEELKPTPEG | | |
| | DLEILLQKWENGECAQKKIIAEKTKIPAVFKIDALN | | |
| | ENKVLVLDTDYKKYLLFCMENSAEPEQSLACQCLV | | |
| | RTPEVDDEALEKFDKALKALPMHIRLSFNPTQLEEQ | | |
| | CHV | | |

Or a sequence having between 80% and 99% homology, its mnRNA and cDNA or showing the activity of the encoded proteins.

Moreover, the Bubalus bubalis polypeptide, expressed, isolated and collected using the systems disclosed can be a mature milk protein having antimicrobial activity, that is at least one of: lactoferrin, lactoperoxidase and lysozyme C, encoded by the following sequences:

| SEQ ID | | | |
|---|---|---|---|
| No. | Sequence | Name | Encodes |
| 13 | ATGAAGCTCTTCGTCCCCGCCCTGCTGTCCCTTGGA | Lactoferrin | AJ005203.1 |
| | GCCCTTGGACTGTGTCTGGCTGCCCCGAGGAAAAA | | |
| | CGTTCGATGGTGTACCATCTCCCAACCCGAGTGGCT | | |
| | CAAATGCCACCGATGGCAGTGGAGGATGAAGAAGC | | |
| | TGGGTGCTCCCTCTATCACCTGTGTGAGGAGGGCCT | | |
| | TTGTCTTGGAATGTATCCGGGCCATCACGGAAAAA | | |
| | AAGGCAGATGCTGTGACCCTGGATGGTGGCATGGT | | |
| | GTTTGAGGCAGGCCTGGACCCCTACAAACTGCGGC | | |
| | CAGTAGCAGCAGAGATCTATGGGACCAAAGAGTCT | | |
| | CCCCAAACCCACTATTATGCTGTGGCTGTCGTCAAA | | |
| | AAGGGCAGCAACTTTCAGCTGGACCAGCTGCAAGG | | |
| | CCGGAATTCCTGCCATACGGGCCTTGGCAGGTCTGC | | |
| | TGGGTGGAACATCCCTATGGGAATCCTTCGCCCGTA | | |
| | CTTGAGCTGGACAGAGTCACTCGAGCCCTTCCAGG | | |
| | GAGCTGTGGCTAAATTCTTCTCTGCCAGCTGTGTTC | | |
| | CCTGCGTTGATAGACAAGCGTACCCCAACCTGTGTC | | |
| | AACTGTGCAAGGGGGAGGGGGAGAACCAGTGTGCC | | |
| | TGCTCCCCCCGGGAACCATACTTCGGCTATTCTGGT | | |
| | GCCTTCAAGTGTCTGCAGGACGGGGCTGGAGACGT | | |
| | GGCTTTTGTCAAGGAGACGACAGTGTTTGAGAACTT | | |
| | GCCAGAGAAGGCTGACAGGGACCAGTATGAGCTTC | | |
| | TCTGCCTAAACAACACTCGGGCACCAGTGGATGCA | | |
| | TTCAAGGAGTGCCACCTGGCCCAGGTCCCTTCTCAT | | |
| | GCTGTCGTGGCCCGAAGTGTGGATGGCAAGGAAGA | | |
| | CTTGATCTGGAAGCTTCTCAGCAAGGCGCAGGAGA | | |
| | AGTTCGGAAAAAACAAGTCTGGGAGCTTCCAGCTC | | |
| | TTTGGCTCTCCACCCGGCCAGAGGGACCTGCTATTC | | |
| | AAAGACTGTGCTCTTGGGTTTTTGAGGATCCCCTCG | | |
| | AAGGTAGATTCGGCACTGTACCTGGGCTCCCGCTAC | | |
| | TTGACCGCCTTGAAAAACCTCAGGGAAACTGCGGA | | |
| | GGAGGTGCAGGCACGGCGCGCGAGGGTCGTGTGGT | | |
| | GCGCGGTGGGACCCGAGGAGCAGAAAAAGTGCCA | | |
| | GCAGTGGAGCCAGCAGAGCGGCCAGATCGTGACCT | | |
| | GTGCCACGGCCTCCACCACCGATGACTGCATCGCCC | | |
| | TGGTGCTGAAAGGGGAAGCGGATGCCCTGAGCTTG | | |
| | GATGGAGGATATATCTACACTGCGGGCAAGTGTGG | | |
| | TCTGGTGCCTGTCCTGGCAGAGAACCGGAAATCCTC | | |
| | CAAACACAGTAGCCTAGATTGTGTGCTGAGACCAA | | |
| | CGGAAGGGTACCTTGCCGTGGCAGTTGTCAAGAAA | | |
| | GCAAATGAGGGGCTCACTTGGAATTCTCTGAAAGG | | |
| | CAAGAAGTCGTGCCACACCGCCGTGGACAGGACTG | | |
| | CAGGCTGGAACATCCCCATGGGCCTGATCGCCAAC | | |
| | CAGACAGGCTCCTGCGCATTTGATGAATTCTTTAGT | | |
| | CAGAGCTGTGCCCCTGGGGCTGACCCGAAATCCAG | | |
| | ACTCTGTGCATTGTGTGCTGGCGATGACCAGGGCCT | | |
| | GGACAAGTGTGTGCCCAACTCTAAGGAGAAGTACT | | |
| | ATGGCTACACCGGGGCTTTCAGGTGCCTGGCTGAG | | |
| | GATGTTGGGGACGTTGCCTTTGTGAAAAATGACAC | | |
| | AGTTTGGGAGAACACGAATGGAGAGAGCACTGCAG | | |
| | ACTGGGCTAAGAACTTGAATCGCGAGGACTTCAGG | | |
| | TTGCTTTGCCTCGATGGCACCAGGAAGCCTGTGACG | | |
| | GAGGCTCAGAGCTGCCACCTGGCGGTGGCCCCGAA | | |
| | TCACGCTGTGGTGTCTTTGAGCGAAAGGGCAGCTCA | | |
| | CGTGGAACAGGTGCTGCTCCACCAGCAGGCTCTGTT | | |
| | TGGGGAAAATGGAAAAAACTGCCCGGACAAATTTT | | |
| | GTTTGTTCAAATCTGAAACCAAAAACCTTCTGTTCA | | |
| | ATGACAACACTGAGTGTCTGGCCAAACTTGGAGGC | | |
| | AGACCAACGTATGAAGAATATTTGGGGACAGAGTA | | |
| | TGTCACAGCCATTGCCAACCTGAAAAAATGCTCAA | | |
| | CCTCCCCGCTTCTGGAAGCCTGCGCCTTCCTGACGA | | |
| | GGTAA | | |
| 14 | MKLFVPALLSLGALGLCLAAPRKNVRWCTISQPEWL | | CAA06441.1 |
| | KCHRWQWRMKKLGAPSITCVRRAFVLECIRAITEKKA | | |
| | DAVTLDGGMVFEAGLDPYKLRPVAAEIYGTKESPQT | | |
| | HYYAVAVVKKGSNFQLDQLQGRNSCHTGLGRSAGW | | |
| | NIPMGILRPYLSWTESLEPFQGAVAKFFSASCVPCVDR | | |
| | QAYPNLCQLCKGEGENQCACSPREPYFGYSGAFKCLQ | | |
| | DGAGDVAFVKETTVFENLPEKADRDQYELLCLNNTR | | |
| | APVDAFKECHLAQVPSHAVVARSVDGKEDLIWKLLS | | |
| | KAQEKFGKNKSGSFQLFGSPPGQRDLLFKDCALGFLRI | | |
| | PSKVDSALYLGSRYLTALKNLRETAEEVQARRARVV | | |
| | WCAVGPEEQKKCQQWSQQSGQIVTCATASTTDDCIA | | |
| | LVLKGEADALSLDGGYIYTAGKCGLVPVLAENRKSSK | | |
| | HSSLDCVLRPTEGYLAVAVVKKANEGLTWNSLKGKK | | |
| | SCHTAVDRTAGWNIPMGLIANQTGSCAFDEFFSQSCA | | |
| | PGADPKSRLCALCAGDDQGLDKCVPNSKEKYYGYTG | | |
| | AFRCLAEDVGDVAFVKNDTVWENTNGESTADWAKN | | |
| | LNREDFRLLCLDGTRKPVTEAQSCHLAVAPNHAVVSL | | |
| | SERAAHVEQVLLHQQALFGENGKNCPDKFCLFKSETK | | |
| | NLLFNDNTECLAKLGGRPTYEEYLGTEYVTAIANLKK | | |
| | CSTSPLLEACAFLTR | | |
| 15 | AGTGTGGGAGGCTGTGGAGTCCGGGCCCCTAATTCT | Lacto- | NM_001290883.1 |
| | CCCTGGAGGCCCAGGGTGAGAGATCCAGCTTATCA | peroxidase | |
| | ACGGCTGAGCAATCCACAAAGTCATGTGGGTAGTT | | |
| | GTTGGCCTGGAAGGCATGCAGCGGGACCTTCGTGA | | |
| | TGTGGGTCTGTCTCCAACTTCCAGTCTTTTTGGCTTC | | |
| | CGTGACCTTATTCGAGGTTGCAGCATCTGACACAAT | | |
| | TGCACAGGCCGCCAGCACCACCACCATCTCTGATGC | | |
| | TGTGAGTAAGGTCAAGATCCAGGTCAACAAGGCCT | | |
| | TCCTGGATTCCCGGACCAGGCTGAAGACGACCTTG | | |
| | AGCTCTGAGGCACCCACCACCCAACAGCTCTCAGA | | |
| | GTACTTCAAGCACGCCAAGGGCCAGACCCGCACGG | | |
| | CCATTCGCAACGGGCAGGTGTGGGAGGAGTCCTTC | | |
| | AAGAGGCTGAGGCGGGACACAACCCTGACCAACGT | | |
| | CACAGACCCTAGCTTGGACTTGACTGCACTCTCCTG | | |
| | GGAGGTGGGCTGCGGTGCCCCGGTTCCTCTGGTGA | | |
| | AATGTGATGAAAACAGCCCTTACCGCACCATCACG | | |
| | GGAGACTGTAATAACAGGAGGAGCCCCGCGCTGGG | | |
| | CGCCGCCAACAGGGCGCTGGCGCGCTGGCTGCCGG | | |
| | CGGAGTACGAGGACGGGCTCGCCCTGCCCTTCGGC | | |
| | TGGACGCAGAGGAAGACGCGCAACGGCTTCCGCGT | | |
| | CCCGCTGGCCCGTGAGGTATCCAACAAAATTGTAG | | |
| | GCTACCTGGACGAAGATGGTGTTCTGGACCAAAAC | | |
| | AGGTCCCTGCTCTTCATGCAGTGGGGTCAAATTGTG | | |
| | GACCACGACCTGGACTTTGCCCCAGAAACGGAACT | | |
| | GGGGAGCAACGAGCACTCTAAAACCCAGTGTGAGG | | |
| | AGTACTGTATCCAGGGAGACAACTGCTTCCCCATCA | | |
| | TGTTCCCGAAAAATGATCCCAAGTTGAAGACTCAA | | |
| | GGGAAATGCATGCCTTTCTTCCGAGCCGGGTTTGTC | | |
| | TGCCCCACTCCACCTTACCAGTCGTTGGCCCGAGAA | | |
| | CAGATCAATGCTGTGACCTCCTTCCTGGACGCCAGC | | |
| | TTAGTGTACGGCTCTGAGCCCAGTCTGGCCAGCCGT | | |
| | CTCCGGAACCTCAGCAGCCCGCTGGGCCTCATGGCT | | |
| | GTCAACCAAGAAGCCTGGGACCACAGGCTGGCCTA | | |
| | CCTGCCCTTTAACAACAAGAAGCCGAGCCCCTGTG | | |
| | AGTTCATCAACACCACTGCCCATGTGCCCTGTTTCC | | |
| | TGGCGGGAGATTTTCGAGCCTCAGAGCAGATCCTG | | |
| | CTGGCCACTGCCCACACCCTCCTTCTCCGGGAGCAC | | |
| | AACCGGCTGGCCAGAGAACTAAAGAAACTCAACCC | | |
| | TCAATGGGATGGAGAGAAGCTCTACCAGGAAGCCC | | |
| | GGAAAATCCTGGGAGCTTTCGTCCAGATTATCACCT | | |
| | TTAGGGACTACCTACCCATTGTGCTAGGTAGTGAGA | | |
| | TGCAGAAGTGGATCCCGCCCTACCAAGGCTATAAT | | |
| | AACTCTGTGGATCCCCGAATTTCCAATGTCTTCACC | | |
| | TTTGCCTTCCGCTTTGGCCACATGGAGGTTCCCTCC | | |
| | ACTGTGTCCCGCCTGGATGAGAATTACCAGCCATGG | | |
| | GGTCCGGAAGCAGAGCTCCCCCTGCACACCCTCTTC | | |
| | TTCAACACCTGGAGGATAATCAAAGATGGTGGAAT | | |
| | TGACCCTCTGGTGCGGGGTCTGCTGGCCAAGAAGTC | | |
| | CAAACTGATGAATCAGGATAAAATGGTGACGAGTG | | |
| | AGCTGCGCAACAAGCTTTTCCAGCCCACTCACAAG | | |
| | ATCCACGGCTTTGACCTGGCTGCTATCAACTTACAG | | |
| | CGTTGCCGGGACCATGGGATGCCTGGGTACAACTC | | |
| | CTGGAGGGGCTTCTGTGGCCTCTCACAGCCCAAGAC | | |
| | GCTGAAGGGGCTGCAGACCGTGCTGAAGAACAAGA | | |
| | TACTGGCTAAGAAGTTAATGGATCTCTACAAGACCC | | |
| | CCGACAACATTGACATCTGGATTGGAGGCAACGCT | | |
| | GAGCCCATGGTAGAAAGGGGCCGGGTGGGGCCTCT | | |
| | CCTGGCCTGCCTCCTAGGGAGGCAATTCCAGCAGAT | | |
| | ACGTGATGGGGACAGGTTCTGGTGGGAGAACCCTG | | |
| | GGGTCTTCACTGAGAAGCAGCGGGACTCTCTACAG | | |
| | AAAATGTCCTTCTCGCGCCTCATCTGTGACAACACC | | |
| | CACATCACGAAGGTCCCGCTGCATGCCTTCCAGGCC | | |
| | AACAACTACCCACATGACTTTGTGGATTGCTCAGCC | | |
| | GTTGATAAGCTGGATCTCTCACCCTGGGCCTCCAGG | | |
| | GAGAATTAGGGGCCCGGACTCCACAGCCTCCCACA | | |
| | CT | | |
| 16 | MWVCLQLPVFLASVTLFEVAASDTIAQAASTTTISDA | | NP_001277812.1 |
| | VSKVKIQVNKAFLDSRTRLKTTLSSEAPTTQQLSEYFK | | |
| | HAKGQTRTAIRNGQVWEESFKRLRRDTTLTNVTDPSL | | |
| | DLTALSWEVGCGAPVPLVKCDENSPYRTITGDCNNRR | | |
| | SPALGAANRALARWLPAEYEDGLALPFGWTQRKTRN | | |
| | GFRVPLAREVSNKIVGYLDEDGVLDQNRSLLFMQWG | | |
| | QIVDHDLDFAPETELGSNEHSKTQCEEYCIQGDNCFPI | | |
| | MFPKNDPKLKTQGKCMPFFRAGFVCPTPPYQSLAREQ | | |
| | INAVTSFLDASLVYGSEPSLASRLRNLSSPLGLMAVNQ | | |
| | EAWDHRLAYLPFNNKKPSPCEFINTTAHVPCFLAGDF | | |
| | RASEQILLATAHTLLLREHNRLARELKKLNPQWDGEK | | |
| | LYQEARKILGAFVQIITFRDYLPIVLGSEMQKWIPPYQ | | |
| | GYNNSVDPRISNVFTFAFRFGHMEVPSTVSRLDENYQ | | |
| | PWGPEAELPLHTLFFNTWRIIKDGGIDPLVRGLLAKKS | | |
| | KLMNQDKMVTSELRNKLFQPTHKIHGFDLAAINLQRC | | |
| | RDHGMPGYNSWRGFCGLSQPKTLKGLQTVLKNKILA | | |
| | KKLMDLYKTPDNIDIWIGGNAEPMVERGRVGPLLACL | | |
| | LGRQFQQIRDGDRFWWENPGVFTEKQRDSLQKMSFS | | |
| | RLICDNTHITKVPLHAFQANNYPHDFVDCSAVDKLDL | | |
| | SPWASREN | | |
| 17 | ATGTTAAATACCAAGTCCAGCTCACCTGGTCAACTT | lysozyme C, | XM_006058377.4 |
| | GGACATTTGGCTTCTGTCAACATGAAGGCTCTCCTT | milk | |
| | ATTGTGGGGCTTCTCCTCCTTTCTGTTGCTGTCCAGG | isozyme | |
| | GCAAGAAATTTGGGAGGTGTGAGCTTGCCAGAACT | | |
| | CTGAAGAAACTTGGATTGGCTGGCTACAAGGGAGT | | |
| | CAGCCTGGCAAACTGGATGTGTTTGGCCAGATGGG | | |
| | AAAGCAATTACAACACACGTGCTACACACTACAAT | | |
| | CGTGGAGACAAAAGCACTGATTATGGGATATTTCA | | |
| | AATCAATAGCCGCTGGTGGTGCAATGATGGCAAAA | | |
| | CCCCAAGAGCAGTTAACGCCTGTGGTATACCCTGCA | | |
| | GCGCTTTGCTGAAAGATGACATCACTCAAGCTGTAA | | |
| | CATGTGCAAAGAGGGTTGTCAGAGATCCACAAGGC | | |
| | ATTAGAGCATGGGTGGCATGGAGAAACAAGTGTCA | | |
| | AAACCGAGACCTCACAAGTTATGTTAAGGGTTGCG | | |
| | GAGTGTAA | | |
| 18 | MLNTKSSSPGQLGHLASVNMKALLIVGLLLLSVAVQG | | XP_006058439.3 |
| | KKFGRCELARTLKKLGLAGYKGVSLANWMCLARWE | | |
| | SNYNTRATHYNRGDKSTDYGIFQINSRWWCNDGKTP | | |
| | RAVNACGIPCSALLKDDITQAVTCAKRVVRDPQGIRA | | |
| | WVAWRNKCQNRDLTSYVKGCGV | | |

Or a sequence having between 80% and 99% homology, its mRNA and cDNA or showing the activity of the encoded proteins.

System 10, can optionally further comprise bioreactor 1014 comprising carrier (1044, not shown) having thereon and being operable to support a plurality of Bubalus bubalis mammary epithelial cells (MECs), adapted to secrete and accumulate a fatty acid, or additionally, or alternatively a (bioreactor e.g., with-) carrier 1045 (not shown) having thereon a recombinant yeast adapted to overproduce and overexpress extra-cellular free fatty acids (FFAs), wherein bioreactor 1014 comprising the Bubalus bubalis MECs, or the recombinant yeast is in liquid communication with mixing tank 100, and a FFA separator 302, FFA separator 302 being in further in liquid communication with mixing tank 100.

Mammary epithelial cells (MECs) secrete milk constituents by several routes. Milk lipid is enveloped by a milk fat globule membrane (MFGM) derived from the apical cell surface, and contains some of its constituent proteins. Soluble milk proteins are secreted by exocytosis. MECs can be cultured directly on scaffolding (e.g., carrier 1044), embedded e.g., in a reconstituted basement membrane that can be further cultured a medium containing lactogenic hormones (e.g., estrogen, progesterone and prolactin).

Additionally, or alternatively, genetically modified yeast lines are genetically engineered in an exemplary implementation, to overproduce and/or overexpress extracellular FFAs. In another exemplary implementation, engineered yeast (e.g., S. cerevisiae) line would include deletions in the following genes: FAA2, FAA1, FAA4 and FAT1 (acyl-CoA synthetase), PXA1 (coding for a subunit of the ABC transporter complex Pxa1-Pxa2 that is responsible for importing long chain fatty acids into the peroxisome) and POX1(fatty acyl-CoA oxidase). It would also include overexpression of DGA1 (diacylglycerol acyltransferase) and TGL3 (triacylglycerol lipase). Accordingly, the recombinant yeast adapted to overproduce, and/or overexpress extracellular free fatty acids (FFAs) comprises a heterologous polynucleotides encoding genes having selective deletions, configured to overexpress extracellular FFAs, comprising deletion in at least one of: FAA2, FAA1, FAA4, FAT1, PXA1, and POX1.

As used herein, the term “genetic engineering”, or “genetically engineered” refer to the creation of a non-natural DNA, protein, or organism that would not normally be found in nature and therefore entails applying human intervention. Genetic engineering can be used to produce an engineered DNA, protein, or organism that was conceived of and created in the laboratory using one or more of the techniques of biotechnology such as molecular biology, protein biochemistry, bacterial transformation, transfection, and plant transformation. For example, genetic engineering can be used to create a chimeric gene comprising at least two DNA molecules heterologous to each other using one or more of the techniques of molecular biology, such as gene cloning, DNA ligation, and DNA synthesis, for example, CRISPR-cas9 system. A chimeric gene may consist of two or more heterologous DNA molecules that are operably linked, such as a protein-coding sequence operably linked to a gene expression element such as a transit peptide-coding sequence or a heterologous promoter. Genetic engineering can be used to create an engineered protein whose polypeptide sequence was created using one or more of the techniques of protein engineering, such as protein design using site-directed mutagenesis and directed evolution using random mutagenesis and DNA shuffling. An engineered protein may have one or more deletions, insertions, or substitutions relative to the coding sequence of the wild-type protein and each deletion, insertion, or substitution may consist of one or more amino acids. In another exemplary implementation, an engineered protein may consist of two heterologous peptides that are operably linked, such as an enzyme operably linked to a transit peptide.

In another exemplary implementation, the genetically engineered yeast line can include deletions in acyl-CoA synthetase genes (Δfaa1 and Δfaa4) and fatty acyl-CoA oxidase (Δpox1), and overexpression of ATP:citrate lyase (ACL), malic enzyme (ME), limitochondrial citrate transporter (Ctp1), malate dehydrogenase (Mdh3), fatty acid synthase genes (FAS1 and FAS2), a truncated thioesterase (′tesA) and enhanced expression of the endogenous acetyl-CoA carboxylase ACC1 by replacing its native promoter with the TEF1 promoter represented in an exemplary implementation by by the sequence:

(SEQ ID. NO. 19)
CAATGCATACTTTGTACGTTCAAAATACAATGCAGTAGATATATTTATG
CATATTACATATAATACATATCACATAGGAAGCAACAGGCGCGTTGGAC
TTTTAATTTTCGAGGACCGCGAATCCTTACATCACACCCAATCCCCCAC
AAGTGATCCCCCACACACCATAGCTTCAAAATGTTTCTACTCCTTTTTT
ACTCTTCCAGATTTTCTCGGACTCCGCGCATCGCCGTACCACTTCAAAA
CACCCAAGCACAGCATACTAAATTTCCCCTCTTTCTTCCTCTAGGGTGT
CGTTAATTACCCGTACTAAAGGTTTGGAAAAGAAAAAAGAGACCGCCTC
GTTTCTTTTTCTTCGTCGAAAAAGGCAATAAAAATTTTTATCACGTTTC
TTTTTCTTGAAAATTTTTTTTTTTGATTTTTTTCTCTTTCGATGACCTC
CCATTGATATTTAAGTTAATAAACGGTCTTCAATTTCTCAAGTTTCAGT
TTCATTTTTCTTGTTCTATTACAACTTTTTTTACTTCTTGCTCATTAGA
AAGAAAGCATAGCAATCTAATCTAAGTTTTAATTACAAA

In the context of the disclosure, the term “promoter” refers to a region of DNA upstream from the translational start codon and which is involved in recognition and binding of RNA polymerase and other proteins to initiate transcription. The term “operably linked” as used herein, refers to linkage of a promoter upstream from a DNA sequence such that the promoter mediates transcription of the DNA sequence (e.g., SEQ ID No.s 1, 3, 5). It is understood that the promoter sequence (e.g., SEQ ID No. 19) also includes transcribed sequences between the transcriptional start and the translational start codon. In an exemplary implementation, various promoters will be selected based on the organism in which the protein is expressed. for example, Tef-1 (see e.g., SEQ ID No. 19) can be used for S. cerevisiae and P. pastoris.

Utilizing oleaginous microorganisms; fungi, yeasts and algae can be adapted in an exemplary implementation to accumulate lipids as much as 20% of their dry cellular weight. Oils/fats accumulated by oleaginous microbes (OM) are gaining significant interest owing to the quality of lipids, which can be used for either food consumption, or fuel purpose. In oleaginous yeasts, lipids are found mainly in the form of neutral lipids, glycolipids, phospholipids, and free fatty acids (FFA). The oleaginous yeasts used in the systems and methods disclosed, can be, for example the genera Yarrowia, Rhodotorula (Rhodosporidium), Lipomyces, Cryptococcus and Trichosporon. Specifically, Yarrowia lipolytica has shown to be a convenient host for industrial processes and as a model organism for investigating lipid synthesis. It is recognized as a generally regarded as safe (GRAS) microorganism, and for this reason Y. lipolytica is used in an exemplary implementation as a host for the production of dietary supplements and nutraceuticals (Caporusso et al., 2021). Wild-type Y. lipolytica grows on a variety of substrates and can accumulate lipids intracellularly to ≥40% of its cell dry weight. Y. lipolytica is known for its pronounced lipolytic and proteolytic activities that is naturally found in foods with high proportions of fat and/or protein, particularly in (fermented) dairy products and meat.

In yet another exemplary implementation, system 10 further comprises bioreactor 1015 comprising carrier 1046 (not shown) having thereon a recombinant yeast comprising heterologous polynucleotides encoding at least one human milk oligosaccharide (HMO), wherein bioreactor 1015 comprising the recombinant yeast is in liquid communication with mixing tank 10, and HMO separator 303, whereby HMO separator 303 is being in further liquid communication with mixing tank 100. For example, the most abundant HMO, 2′-fucosyllactose (2′-FL), can be produced in system 10 disclosed, by genetically engineered S. cerevisiae cell factory. 2′-FL synthesis and secretion in engineered S. cerevisiae, is achieved by transfecting the encoding DNA of the organism with four heterologous metabolic components: a lactose transporter, a GDP-L fucose synthetic pathway, α-1,2-fucosyltransferase and a 2′-FL transporter. In an exemplary implementation heterologous α-1,2-fucosyltransferase is expressed in Bacillus cereus (FutBc) coding nucleotide and deletion of gal80, where the resulting strain produced 19.56 g/L extracellular and 7.07 g/L intracellular. Accordingly, and in an exemplary implementation, the recombinant yeast's heterologous polynucleotide encodes a deletion of gal80 gene.

In an exemplary implementation, secreted proteins, and optionally fatty acids or HMOs enter the mixing tank 100 through molecular weight cutoff membranes (5-200 Kda) 102j that will prevent the producing organisms and cells from entering mixing tank 100 as well. This will be achieved via array of valves 103p according to the desired recipe for specific products, i.e., standardized milk, whey or crème. Furthermore, a protein purification step may be implemented, whereby separation is done using for example, size exclusion membrane and/or hollow fiber tangential flow filtration (TFF) and may also include protein isolation by binding to an affinity column. For example, His-tag purification columns (immobilized metal affinity chromatography (IMAC) columns such as, Ni-NTA Agarose column). Accordingly and in an exemplary implementation, each i^{th} bioreactor 1011, 1012, 1013, is in further communication with protein purification module 301, operable to selectively isolate a predetermined protein secreted by each i^{th} bioreactor 1011, 1012, 1013, whereby protein purification module 301 comprises at least one of: a size exclusion column, a hollow fiber tangential flow filtration (TFF) column, and an affinity column 3010.

An additional step may further comprise the extraction of fatty acids and HMOs. As for fatty acids—in the food industry, ethanol and hexane are widely used as low-toxicity solvents for lipid extraction. Green solvents such as bio-derived solvents, ionic liquids and deep eutectic solvents are employed in an exemplary implementation for the extraction of oil from oleaginous microbes. These green solvents are eco-friendly, low in energy and solvent consumptions and display higher efficiency in product formations. Additional green extraction techniques can comprise enzyme assisted extraction (AEE), microwave assisted extraction (MAE) and ultrasound assisted extraction (UAE) (Kumar et al., 2021). Extraction of fatty acids will be followed by their supplement and admixing into mixing tank 100.

Furthermore, a combination of a cationic ion exchanger treatment, an anionic ion exchanger treatment, and a nanofiltration and/or electrodialysis step, allows efficient purification of large quantities of neutral HMOs at high purity and without the need of a chromatographic separation. The purified HMOs may be obtained in solid form post processing by spray drying, as crystalline material or as sterile filtered concentrate. Like the FFAs, separation and purification of HMOs will be followed by their supplement into mixing tank 100.

Collection of purified proteins, as a stand-alone product composed of a mixture of proteins or a single protein according to demand. Purified proteins can also be used as additives and can be added to the mixing tank according to the specificities of each product (i.e., standardized milk, crème or whey).

In an exemplary implementation, system 10 further comprises additive container 500 in liquid communication with mixing tank 100, operable to selectably provide a predetermined additive into the mixing tank. The addition of additives is done according to the requirement per specific product (milk, crème, whey). This may include sugars (plant source including sugar-beet, agave, carrot), vitamins, minerals (including Calcium, phosphorus, sodium and potassium), plant proteins, amino acids, antioxidants, plant-source fatty acids and the like (alternatively or in addition to fatty acids obtained from mammary gland cells or by cell factory yeast lines or oleaginous microorganisms). Moreover, sugar solution used in certain exemplary implementations, can further comprise sugar alcohols, for example: adonitol, allitol, altritol, arabinitol, dulcitol, erythritol, glycerol, iditol, inositol, isomalt, lactitol, maltitol, mannitol, perseitol, ribitol, rhamnitol, sorbitol, threitol or xylitol. Moreover, the sugar solution used in certain exemplary implementations, can comprise indigestible sugars (iS), such as for example: difructose anhydride (DFA) III, fructooligosaccharides (FOS), xylooligosaccharides (XOS), mannanoligosaccharides (MOS), galactooligosaccharides (GOS), and the like. Use of the sugar solutions comprising the sugar alcohols, and iS, in combination with other sugars, can be used to produce low-calorie cultured Buffalo milk.

In additional step, the milk, protein or milk product(s) are collected using collection vessels 601, 602, 603, and finally, vat 800 for post-processing operations. Other post-processing operations can be implemented. The production of dairy products (i.e., pasteurized milk, different cheeses, yogurt, butter etc.) can involve various downstream processes such as, homogenization, pasteurization, fermentation, coagulation etc. Each dairy product will be handled and processed with its own unique set of requirements. In another exemplary implementation production of rennet via yeast/bacteria/fungi/buffalo mammary epithelial cell line can be achieved, whereby Buffalo chymosin (rennet) will be accumulated, purified, and used as a coagulation enzyme which is important to the process of (e.g., Mozzarella) cheese making.

The term “homology” as used herein refers to a percentage of identity between two polynucleotides or polypeptide moieties. The homology between sequences from one moiety to another moiety may be determined by known techniques. For example, the homology may be determined by directly aligning parameters of sequence information between two polynucleotide molecules or two polypeptide molecules, such as score, identity, and similarity, etc., using a computer program that sorts sequence information and is readily available (e.g., BLAST 2.0). Further, the homology between polynucleotides may be determined by hybridization of the polynucleotide under a condition in which a stable double strand is formed between homologous regions, followed by degradation by a single-strand-specific nuclease to determine a size of the degraded fragment.

Further, as long as a protein has an activity corresponding to a Buffalo milk protein consisting of the detailed amino acid sequence disclosed, it is possible to add a nonsense sequence before and after the amino acid sequence, or to include a naturally occurring mutation or a silent mutation thereof. In addition, polypeptides having a Buffalo milk protein activity may also be included without limitation as a polypeptide encoded by a polynucleotide that is hybridized with a complementary sequence to all or a part of the nucleotide sequence encoding a probe that is able to be prepared from a known gene sequence, for example, the Buffalo milk nucleotide sequences provided herein, under stringent condition. The term “stringent condition” as used herein means a condition that allows specific hybridization between polynucleotides. The condition depends on a length of the polynucleotide and a degree of complementarity. Parameters thereof are well known in the art and are specifically described in the document (e.g., J. Sambrook et al., supra). For example, the stringent condition may list a condition for hybridizing genes to each other each having high homology of 80%, 90%, 95%, 97%, or 99% or more, a condition for not hybridizing genes to each other each having homology lower than that, or a general washing condition of southern hybridization, i.e., a condition for washing once, specifically two to three times at a salt concentration and a temperature such as 60° C., 1×SSC, 0.1% SDS, specifically, 0° C., 0.1×SSC, 0.1% SDS, and more specifically, 68° C., 0.1×SSC, 0.1% SDS. The probe used in the hybridization may be a part of the complementary sequence of the base sequence. Such a probe may be constructed by a PCR using a gene fragment including the base sequence as a template, by utilizing an oligonucleotide prepared based on the known sequence as a primer. Further, those skilled in the art may adjust the temperature and the salt concentration of the wash solution as needed depending on factors such as a length of the probe (or amplicon).

The processes disclosed, are implemented in an exemplary implementation illustrated for example in FIG. 1. Bioreactors 1011 1012, and 1013 are generally configured for protein production chambers that allow regulated and controlled protein entrance to mixing tank 100 (production organism are unable to enter the main tank due to, e.g., a size exclusion membrane(s) 102j) via an array of valves 103p. Alternatively, in batch processes the proteins secreted by the microorganisms may be separated by stages of centrifugation (following a heat shock, utilizing the expressed proteins' thermal stability, to remove micro organisms) and protein isolation by binding of protein to His-tag purification columns (whereby the proteins will be adapted to express His-tag).

Collection vessel 601, 602, 603 can each further have stirrer 107n, as well as a plurality of in-line sensors 106q.

Accordingly, the methods implemented using systems 10 disclosed are configured in certain exemplary implementations, with plurality of in-line sensors 106q operable to analyze a plurality of physico-chemical parameters and provide a central processing module (CPM 700, not shown) included in the system, with the parameters in real time. These plurality of physico-chemical parameters can be, for example at least two of: temperature, pressure, pH, dynamic viscosity, complex viscosity, etc. The real-time measurement can then be used to control flow valves 103j, as well as the residence time of each unit operation and provide simultaneous control.

As further illustrated in FIG. 1, mixing tank 100 can be operated at temperatures of between about 10° C. and about 90° C.

In the context of the disclosure, the term “operable” means the system and/or the device (e.g., the nutrient dispensing pump) and/or the program, or a certain element, component or step is/are fully functional sized, adapted and calibrated, comprising elements for, having the proper internal dimension to accommodate, and meets applicable operability requirements to perform a recited function when activated, coupled or implemented, regardless of being powered or not, coupled, implemented, effected, actuated, realized or when an executable program is executed by at least one processor associated with the system, method, and/or the device.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All ranges disclosed herein are inclusive of the endpoints, and the endpoints are independently combinable with each other. “Combination” is inclusive of blends, mixtures, alloys, reaction products, and the like. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the chamber(s) includes one or more chamber). Reference throughout the specification to “one implementation”, “another implementation”, “an exemplary implementation,”, and so forth, when present, means that a particular element (e.g., feature, structure, and/or characteristic) described in connection with the implementation is included in at least one implementation described herein, and may or may not be present in other implementations. In addition, it is to be understood that the described elements may be combined in any suitable manner in the various implementations.

All ranges disclosed herein are inclusive of the endpoints, and the endpoints are independently combinable with each other. Furthermore, the terms “first,” “second,” and the like, herein do not denote any order, quantity, or importance, but rather are used to denote one element from another.

The term “module,” as used herein, means, but is not limited to, a software or hardware component, such as a field programmable gate array (FPGA) or an application specific integrated circuit (ASIC), or o combination of components which are configured, together, to perform certain tasks. A module may advantageously be configured to reside on an addressable storage medium and configured to execute on one or more processors. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables as well as pumps, conduits, valves and containers. The functionality provided for in the components and modules may be combined into fewer components and modules or further separated into additional components and modules.

Likewise, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. For example, “about” as used herein mean a reasonable amount of deviation of the modified term such that the end result is not significantly changed. These terms of degree should be construed as including a deviation of at least ±5% or at least ±10%, for example at least ±25% of the modified term if this deviation would not negate the meaning of the word it modifies.

Accordingly, and in an exemplary implementation, provided herein is a system for producing Bubalus bubalis milk product, the system comprising: a plurality of bioreactors, each having a proximal end and a distal end, each bioreactor further containing the at least one of recombinant: a yeast, a bacterium, a fungus, and an algae comprising heterologous polynucleotides encoding a Bubalus bubalis polypeptide, wherein each bioreactor is further being in liquid communication with a mixing tank; and a plurality of collection receptacle, each collection receptacle associated with a product-specific bioreactor combination, wherein (i) the Bubalus bubalis polypeptide is at least one of: a milk protein, a whey protein, and an antimicrobial protein (ii) the milk protein being: αS1-casein (αS1-CN), and/or αS2-casein (αS2-CN), and/or β-casein (β-CN), and/or and κ-casein (κ-CN) and/or their combination, (iii) the whey protein being: β-lactoglobulin (β-LGB) and/or α-lactalbumin B (α-LAB), and/or both, (iv) the antimicrobial protein is at least one of: lactoferrin, lactoperoxidase and lysozyme C, wherein (v) the system further comprises a bioreactor comprising a carrier having thereon Bubalus bubalis mammary epithelial cells (MECs), adapted to secrete and accumulate fatty acids, or a carrier having thereon a recombinant yeast adapted to overproduce extra-cellular free fatty acids (FFAs), wherein the bioreactor comprising the Bubalus bubalis MECs, or the recombinant yeast is in liquid communication with the mixing tank, and a FFA separator, the FFA separator being in further in liquid communication with the mixing tank, the system (vi) further comprising a bioreactor comprising a carrier having thereon a recombinant yeast comprising heterologous polynucleotides encoding at least one human milk oligosaccharide (HMO), wherein the bioreactor comprising the recombinant yeast is in liquid communication with the mixing tank, and a HMO separator, the HMO separator being in further liquid communication with the mixing tank, wherein (vii) the recombinant yeast adapted to overproduce extracellular free fatty acids (FFAs, meaning that the amount of FFAs produced from the recombinant yeast will be greater than the amount of FFAs produced from any or all of the wild type yeast) comprises a heterologous polynucleotides encoding genes having selective deletions, configured to overexpress extracellular FFAs, (viii) the heterologous polynucleotides encoding genes being: FAA2, and/or FAA1, and/or FAA4, and/or FAT1, and/or PXA1, and/or POX1, and/or their combination, furthermore (ix) the heterologous polynucleotides is adapted to overexpress: DGA1 (diacylglycerol acyltransferase) and/or TGL3 (triacylglycerol lipase), or (x) the heterologous polynucleotides is adapted to overexpress: gene of ATP:citrate lyase (ACL), and/or malic enzyme (ME), and/or limitochondrial citrate transporter (Ctp1), and/or malate dehydrogenase (Mdh3), and/or fatty acid synthase genes (FAS1 and FAS2), and/or a truncated thioesterase ('tesA), and/or endogenous acetyl-CoA carboxylase (ACC1), and/or their combination, wherein (xi) the recombinant yeast's heterologous polynucleotide encodes a lactose transporter, a GDP-L fucose synthetic pathway, α-1,2-fucosyltransferase and a 2′-FL transporter, (xii) encoding a deletion of gal80 gene, wherein (xiii) each bioreactor is in further communication (either directly, or through an intermediate member or module) with a protein purification module, operable to selectively isolate a predetermined protein, (xiv) the protein purification module comprises at least one of: a size exclusion column, a hollow fiber tangential flow filtration (TFF) column, and an affinity column, the system (xv) further comprising an additive container in liquid communication with the mixing tank, operable to selectably provide a predetermined additive into the mixing tank, and wherein (xvi) the predetermined additive is sugars, and/or vitamins, and/or minerals, and/or plant proteins, and/or amino acids, and/or antioxidants, and/or plant-source fatty acids, and/or their combination.

Although the foregoing disclosure for methods, systems and compositions for producing Buffalo milk products. More specifically, for continuously, batch-wise and semi-continuously using transformed/transfected yeast and/or fungi and/or bacteria and/or algea to express and/or secrete Bubalus bubalis Casein, Whey protein and additional proteins, collecting the expressed product and using it for producing various products, which has been described in terms of some implementations, other implementations will be apparent to those of ordinary skill in the art from the disclosure herein. Moreover, the described implementations have been presented by way of example only, and are not intended to limit the scope of the inventions. Indeed, the novel methods, programs, libraries and systems described herein may be embodied in a variety of other forms without departing from the spirit thereof. Accordingly, other combinations, omissions, substitutions and modifications will be apparent to the skilled artisan in view of the disclosure herein.

1. A system for producing Bubalus bubalis milk product, the system comprising:
a. a plurality of bioreactors, each bioreactor having a proximal end and a distal end, each bioreactor further containing the at least one of recombinant: a yeast, a bacterium, a fungus, and an algae, each comprising heterologous polynucleotides encoding a Bubalus bubalis polypeptide, wherein:
i. each bioreactor is further being in liquid communication with a mixing tank;
ii. at least one bioreactor of the plurality of bioreactors being in liquid communication with the mixing tank, and further comprising a carrier having thereon a recombinant yeast comprising heterologous polynucleotides encoding at least one human milk oligosaccharide (HMO), a lactose transporter, a GDP-L fucose synthetic pathway, α-1,2-fucosyltransferase, a 2′-FL transporter, and a deletion of gal80 gene; and
iii. a HMO separator, the HMO separator being in further liquid communication with the mixing tank; and
b. a plurality of collection receptacle, each collection receptacle associated with a product-specific bioreactor combination.

2. The system of claim 1, wherein the Bubalus bubalis polypeptide is at least one of: a milk protein, a whey protein, and an antimicrobial protein.

3. The system of claim 2, wherein the milk protein is at least one of: αS1-casein (αS1-CN), αS2-casein (αS2-CN), β-casein (β-CN), and κ-casein (κ-CN).

4. The system of claim 2, wherein the whey protein is at least one of: β-lactoglobulin (β-LGB) and α-lactalbumin B (α-LAB).

5. The system of claim 2, wherein the antimicrobial protein is at least one of: lactoferrin, lactoperoxidase and lysozyme C.

6. The system of claim 1, further comprising a bioreactor comprising a carrier having thereon Bubalus bubalis mammary epithelial cells (MECs), adapted to secrete and accumulate fatty acids, or a carrier having thereon a recombinant yeast adapted to overproduce extra-cellular free fatty acids (FFAs), wherein the bioreactor comprising the Bubalus bubalis MECs, or the recombinant yeast is in liquid communication with the mixing tank, and a FFA separator, the FFA separator being in further in liquid communication with the mixing tank.

7. (canceled)

8. The system of claim 1, wherein the recombinant yeast adapted to overproduce extracellular free fatty acids (FFAs) comprises a heterologous polynucleotides encoding genes having selective deletions, configured to overexpress extracellular FFAs.

9. The system of claim 8, comprising deletion in at least one of: FAA2, FAA1, FAA4, FAT1, PXA1, and POX1.

10. The system of claim 8, wherein the heterologous polynucleotides is adapted to overexpress at least one of: DGA1 (diacylglycerol acyltransferase) and TGL3 (triacylglycerol lipase).

11. The system of claim 8, wherein the heterologous polynucleotides is adapted to overexpress at least one gene of ATP:citrate lyase (ACL), malic enzyme (ME), limitochondrial citrate transporter (Ctp1), malate dehydrogenase (Mdh3), fatty acid synthase genes (FAS1 and FAS2), a truncated thioesterase ('tesA), and endogenous acetyl-CoA carboxylase (ACC1).

12. (canceled)

13. (canceled)

14. The system of claim 1, wherein each bioreactor is in further communication with a protein purification module, operable to selectively isolate a predetermined protein.

15. The system of claim 14, wherein the protein purification module comprises at least one of: a size exclusion column, a hollow fiber tangential flow filtration (TFF) column, and an affinity column.

16. The system of claim 1, further comprising an additive container in liquid communication with the mixing tank, operable to selectably provide a predetermined additive into the mixing tank.

17. The system of claim 16, wherein the predetermined additive is at least one of: sugars, vitamins, minerals, plant proteins, amino acids, antioxidants, and plant-source fatty acids.
